(19) United States
(12) Patent Application Publication
(10) Pub. No.: US 2006/0246581 A1
(43) Pub. Date: Nov. 2, 2006

(54) Microorganism culturing apparatus and method of inserting bag into culturing apparatus

(76) Inventor: Fujihiko Tomita, Shizuoka (JP)

Correspondence Address: YAMAHA HATSUDOKI KABUSHIKI KAISHA;C/O KEATING & BENNETT, LLP, 8180 GREENSBORO DRIVE, SUITE 850, MCLEAN, VA, 22102, US

(21) Appl. No.: 10/564,564

(22) PCT Filed: Jul. 1, 2004
(86) PCT No.: PCT/JP04/09321

§ 371 Date: Jun. 14, 2006

(30) Foreign Application Priority Data
Aug. 29, 2003 (JP) 2003-209725

(51) Int. Cl. C12M 1/24

(52) U.S. Cl. 435/304.1; 435/243; 435/292.1; 435/809

(57) ABSTRACT

A microorganism culturing apparatus that is capable of minimizing multiplication of unwanted miscellaneous germs and repeatedly producing the same culturing space includes a transparent bag that defines a container. A gas introduction tube and gas supply tubes are provided in the bag. The bag filled with a culture solution is disposed in a box so that the box holds the bag in an approximately rectangular configuration. The box is made up of a framework having a low-profile, substantially parallelepiped shaped configuration, and flat, transparent plates removably fixed to front and back sides of the framework.

### BACKGROUND OF THE INVENTION

1. Field of the Invention

The present invention relates to a microorganism culturing apparatus.

2. Description of the Related Art

Conventional techniques for a microorganism culturing apparatus are disclosed in Patent Documents 1 to 4 described in more detail below. Patent Document 1 (WO 99/50384 brochure) describes a culturing apparatus using a sealed dome-shaped container for holding a culture solution, with the container having a CO2 gas introducing section and a culture solution discharge section.

Patent Document 2 (JP-A-H10-150974) describes a culture container made of a flattened box with a pair of transparent (side) plates (2 cm or less apart from each other). The top side of the culture container is covered with a lid and tightly sealed with a sealing member. The culture container is also arranged such that a gas supplied from under the culture container is let out from the top side.

Patent Document 3 (JP-A-2000-139444) describes a culturing apparatus including a pair of transparent plates and a supporting framework. This culturing apparatus is constructed such that the paired transparent plates are disposed parallel to each other with a predetermined space in between, with their peripheries fixed to the supporting framework, so that the peripheries are held with the supporting framework in a tightly sealed state. Within the supporting framework are provided supply and discharge flow passages for a culture solution, and supply and discharge flow passages for temperature control water. A gas supply tube for supplying a gas to the culture solution is routed in the culture solution supply flow passage. The document also describes a method for fixing together the transparent plate peripheries and the supporting framework using screws or clamps.

Patent Document 3 also describes an arrangement in which the bottom surface of the culturing apparatus is defined by a pair of surfaces that are not horizontal but sloped toward the center of the horizontal dimension (the distance between a pair of transparent plates) and a gas is supplied from the center. One embodiment of the arrangement is also described in which a culture solution supply flow passage is provided at the lower end of the bottom surface defined by the paired slope surfaces, and a gas supply tube is routed in the supply flow passage. This helps algae, settling when gas supply is stopped, to collect in the center, and also helps algae move up when gas supply is resumed.

Patent Document 4 (JP-A-H06-209757) describes a microorganism culturing apparatus in which a transparent, flexible culture cell made of a material such as polyethylene is supported with a vertical supporting member, and a transportation means is provided to transport biomass retrieved from the culture cell. This document also describes a culture cell as an embodiment in which a large number of culture cells, each having an elliptical shape in cross section, are disposed along the direction of the major axis of the ellipsoid. The document also describes that the culture cell is made by welding together two polyethylene plates face to face.

By the way, in order to culture microorganisms efficiently, it is necessary to prevent the amount of light from becoming insufficient and prevent miscellaneous microbes from growing by regularly cleaning the interior of the culture container and removing foreign objects and miscellaneous microbes adhering to the culture container. However, the conventional techniques mentioned above (the culturing apparatuses described in Patent Documents 1 to 4) leave some room for improvement regarding the facility in cleaning the interior of the culture container. In particular, for the culturing apparatus described in Patent Document 3, because of its structural complexity, cleaning the apparatus in an assembled state is very difficult. Even cleaning after disassembly is cumbersome because there are too many component contact surfaces.

Patent Document 4 does not describe how to clean the culture cell and cleaning the culture cell is not envisaged. Because the microorganism culturing apparatus of Patent Document 4 is constructed to have a flexible culture cell supported by a vertical supporting member, it is hard to create the same culture space every time the apparatus is disassembled, cleaned, and re-assembled.

### SUMMARY OF THE INVENTION

In order to overcome the problems described above, preferred embodiments of the present invention provide a culturing apparatus that is capable of restricting growth of miscellaneous microbes in the culturing solution and that is also capable of repeatedly forming the same culture space.

According to a preferred embodiment of the present invention, a culturing apparatus includes a microorganism, a culturing solution for culturing the microorganism, a container for holding the culturing solution, and a support for accommodating the container, wherein the container maintains a predetermined shape due to being supported by the support. Because the culturing apparatus is constructed such that the container filled with the culturing solution is supported by the support, it is possible to finish a cycle of culturing work by replacing the container without cleaning the support (for example, a box structure). In other words, work is saved because the work of cleaning the culturing apparatus can be replaced with the work of replacing the culturing apparatus. Further, because the support of the culturing apparatus does not need to be watertight but only the container needs to be watertight, the culturing apparatus has a lower cost.

Furthermore, because the container maintains a constant shape due to being held by the support, it is possible to culture the microorganism in the same culturing space even if disassembling and assembling are repeated. This enables operations under unchanged culturing conditions, and makes the culturing apparatus suitable for industrial production. Furthermore, if the container is a bag which does not take a definite shape, work is saved when the bag is replaced by putting the bag into the support and taking the bag out of the support.

The support that is constructed to maintain a predetermined shape of the container includes a bottom member defining a bottom surface and side plates defining side surfaces. The side plates and the container preferably have transparent portions, respectively, and the transparent portions of the side plates and the transparent portions of the container overlap each other.

The side plates are preferably flat plates, and at least one pair of flat surfaces thereof oppose each other. Because the flat plates of the culturing apparatus are disposed opposite to each other so as to face each other, a light path is kept constant and an incident light state in the culturing apparatus is even, so that culturing is carried out efficiently under even culturing conditions.

The support preferably includes side plates for defining side surfaces thereof and a framework for supporting the side plates, and the end portions of the side plates preferably have attachment portions for engaging with the framework and are removably fixed to the framework. With this culturing apparatus, because the framework and the side plates are removably fixed, the side plates may be easily removed by disengaging the framework and the side plates from each other, so that the side plates may be cleaned easily. Similarly, the assembly work may be done easily. For example, even if the amount of light becomes insufficient due to a decrease in a light transmission rate of the side plate if dirt, dust or the like adheres to the side plate, when water is poured on the side plate for temperature control of the culturing solution, satisfactory cleaning can be performed without disassembling the entire culturing apparatus.

The culturing apparatus also preferably includes posts disposed upright with both ends fixed to the framework such that the side plates are squeezed between the pressing members and the posts, and the pressing members are fixed to the posts. With this culturing apparatus, as the fitting portions of the side plates pressed with the entire surfaces of the pressing members, the side plates are fixed firmly to the framework.

The side plates are preferably flat plates disposed end to end in the longitudinal direction of the framework, and the flat plates are interconnected through the posts end to end in the longitudinal direction of the framework, with adjacent two ends of the flat plates fixed to one post with one pressing member. With this culturing apparatus, as the ends of two adjacent flat plates are fixed with one pressing member to one post, it is possible to reduce the number of the pressing members and the posts and also save space in comparison with an arrangement in which the posts are provided one for each flat plate. Further, because the side plates are the plural number of flat plates, the weight of a single plate is reduced. Therefore, the work of disassembly, cleaning, and assembly for the maintenance of the culturing apparatus is simplified and facilitated. Furthermore, the ability to place a plurality of culturing spaces parallel to each other in a single framework makes it possible to carry out both mass production and small-quantity, multi-kind production within a limited space.

The container preferably includes a gas introducing tube for introducing gas into the culturing solution. This culturing apparatus with a gas introducing tube provided in the container makes it possible to increase the amount of gas dissolved in the culturing solution, so that culturing is carried out at high density and with high efficiency. Further, if the container is a bag, as the bag holding the culturing solution is held in a predetermined shape as supported by the support, the gas in the culturing solution held in the bag exists in a favorably dispersed state.

The side plates are preferably supported for rotation about their bottom ends in the state of fixation between the attachment portions and the framework being released. With this culturing apparatus, because the side plates are supported for rotation about their lower ends, the distance between the framework and the side plates may be widened easily, so that the side plates may be cleaned easily without dismantling the framework. Further, the framework does not require any assembly work.

The ends of the side plates on the upper side of the framework are preferably connected through wires to the upper portion side of the framework. This culturing apparatus with the side plates and the framework being interconnected through the wires makes it possible to prevent glass plates from being broken when they are opened too widely or too rapidly. A stopper is preferably provided at the bottom portion to restrict the rotation range of the side plates. Thus, this culturing apparatus provided with the stopper makes it possible to prevent glass plates from being broken when they are opened too widely or too rapidly.

The framework and the side plates are preferably movable relative to each other in the state of fixation between the attachment portions and the framework being released, and the relative motion produces a clearance between the upper portion of the framework and the ends of the side plates on the upper portion side of the framework.

The clearance being produced between the framework and the side plates at the upper portion of the culturing apparatus facilitates cleaning by inserting cleaning tools from above into the apparatus to clean the side plates. Further, while the conventional culturing apparatus requires dismantling of the framework for opening the upper portion of the apparatus widely, the apparatus according to preferred embodiments of the present invention does not require it.

According to another preferred embodiment of the present invention, a culturing apparatus for culturing microorganisms in a culturing solution includes side plates defining side surfaces to define a culturing space, and a bottom portion for defining the bottom of the culturing apparatus to accommodate the culturing solution, wherein the bottom portion has a convex shape directed vertically down toward the center of the width, and a gas introduction tube for introducing a gas into the culturing solution is disposed above the bottom portion at the lower end of the convex portion.

This culturing apparatus in which the gas introduction tube is disposed above the bottom portion at the lower end of the convex-shaped portion makes it possible that, even if a microorganism that has collected in the convex portion further collects in its lowermost portion and further settles on the entire bottom portion, the passage for the gas flowing into the culturing solution cannot be clogged unless the entire flow passage is filled up with the microorganism. Further, the microorganism that settles when gas supply is temporarily stopped does not remain in the corner between the bottom portion and the sides, and is likely to move up when gas supply is resumed.

In contrast, in the case of a gas introduction tube for introducing gas into the culturing solution being disposed below the bottom portion at the lower end of the convex portion, if the microorganism, even in a small amount, settles and collects in the lowermost portion due to the slope of the bottom plate and forms a deposit, there is a possibility that the gas flow passage toward the culturing solution is clogged.

The gas introduction tube preferably has holes along the entire circumference of a substantially circular cross-section thereof.

The culturing apparatus with the gas introduction tube provided with holes along the entire circumference of its substantially circular cross-section makes it possible to reduce gas flow rate per hole in comparison with an arrangement in which holes are provided along only a portion of the substantially circular cross-section. As a result, it is possible to maintain gas bubbles in minute size by restricting recombination of the gas bubbles. It is also possible to reduce the gas bubble diameter by reducing the hole diameter. Therefore, minute bubbles are obtained in a stabilized manner to increase the gas bubble surface area per unit volume of the gas. This results in an increase in the diffusion speed of useful gas ingredients such as oxygen and carbon dioxide into the culturing solution. Therefore, dissolved amounts of oxygen and carbon dioxide necessary for the growth of the microorganism can be easily fixed.

Further, as a large amount of the microorganism adheres to the bubble surfaces and is carried toward the upper portion of the culturing solution against gravitational settling, it is possible to diffuse the microorganism uniformly in the culturing space at a low flow rate. It is also possible to reduce manufacturing costs by lowering the gas flow rate. Therefore, through the use of the culturing apparatus of preferred embodiments of the present invention, it is possible to carry out microorganism culture at high efficiency and low cost.

The container of the culturing apparatus preferably includes a gas introduction tube having minute holes along the entire circumference of its substantially circular cross-section and a gas supply tube for supplying gas to the gas introduction tube, and has an opening at only one position for putting in a microorganism and a culturing solution, with one end of the gas supply tube connected to the gas introduction tube and with the other end thereof extending out through the opening.

The container is preferably a substantially rectangular bag having longer sides and shorter sides, the opening is provided at one end portion of the shorter side of the rectangle, the gas introduction tube is disposed at the other end portion of the shorter side of the rectangle along the longer side of the rectangle, and shaft passage members for letting a shaft pass through are provided along the longer side on the side the opening is provided.

To solve the problems with conventional devices described above, a preferred embodiment of the present invention provides a microorganism culturing apparatus including a container for holding a microorganism and a culturing solution, and a gas introduction tube for introducing a gas into the culturing solution in the container, wherein the container is a bag, the gas introduction tube is disposed in the bag, and the bag is held in a predetermined shape by being supported by a support.

To solve the problems with conventional devices described above, a preferred embodiment of the present invention provides a microorganism culturing apparatus including a container for holding a microorganism and a culturing solution, and a gas introduction tube for introducing a gas into the culturing solution in the container, wherein the container is a transparent bag, the gas introduction tube is disposed in the bag, and the bag filled with the culturing solution is held in an approximately parallelepiped shape by being supported by a support, the support has first and second vertical surface members constituting two pairs of vertical surfaces, the distance between the first vertical surface members is smaller than the distance between the second vertical surface members, and the first vertical surface members are made of a transparent material.

With the culturing apparatuses of the various preferred embodiments of the present invention, because the container is a bag, the container may be discarded after use. Therefore, the process of cleaning the interior of the container of the culturing apparatus every time culture is carried out may be omitted. Further, preparing a sealed bag is easier and lower in cost than preparing a sealed box. The framework for supporting the bag need not be in a sealed state. Therefore, the culturing apparatus of preferred embodiments of the present invention lowers manufacturing cost. Furthermore, making the bag and the vertical surface members transparent makes it possible to use the culturing apparatus for culturing microorganisms by photosynthesis.

In the culturing apparatuses of preferred embodiments of the present invention, it is preferable that the first vertical surface members are made of transparent flat plates. This makes it easier to keep the light path more stabilized in comparison with the case in which the first vertical surface members are made of mesh or lattice. In the culturing apparatuses of preferred embodiments of the present invention, it is preferable that the support is a box structure including a framework and fixing members, with the framework including transparent flat plates defining the first vertical surface members, a pair of beam members defining the top surface of the parallelepiped and being arranged substantially parallel to the vertical surfaces of the first vertical surface members, a floor member serving as the lower surface of the parallelepiped, and the second vertical surface members, with the fixing members removably fixing the flat plates to the framework, with the beam members and the floor member having engagement portions for making the flat plates, paired through a predetermined spacing, engage with the framework.

This makes it possible to easily remove the flat plates from the framework and clean, as the flat plates are required to be cleaned and kept highly transparent. Further, making the support to have the box structure including the framework and the flat plates for supporting the bag makes it possible to support the bag securely and to facilitate disassembly and assembly of the culturing apparatus. Further, because the box structure is obtained by fixing and engaging the paired flat plates with the beam members and the floor member of the framework, it is possible to easily change the light path by providing a plurality of engagement portions in the shorter-side direction of the beam member and floor member, and by preparing flat plates of different dimensions of attachment portions to the beam members and the floor member.

In the culturing apparatuses of preferred embodiments of the present invention, it is preferable that the first vertical surface members are flat plates separated along the longer-side direction of the beam member and the floor member, and the plurality of flat plates are interconnected through posts with both ends thereof fixed to the beam members and the floor member and disposed between the flat plates adjacent in the longer-side direction, pressing members for pressing the mutually adjacent flat plates from outside, and fixing members for removably fixing the pressing members and the posts.

With the above-described construction, in case the total area of the first vertical surfaces remains unchanged, the area of a single flat plate is reduced by the use of a plurality of flat plates in place of a single flat plate for the first vertical surfaces, and handling the flat plates is facilitated. In the culturing apparatuses of preferred embodiments of the present invention, it is preferable that, in the state of the fixing with the fixing members released, the flat plate is supported to be freely rotatable relative to the floor member about the lower end of the flat plate. In this case, it is preferable to interconnect the flat plate and the beam member through a linear member to permit rotation. It is also preferable to provide a stopper to restrict the rotary range of the flat plate.

In the culturing apparatuses of preferred embodiments of the present invention, it is preferable that the gas introduction tube is provided with minute holes along its entire substantially circular cross-section, a bottom surface member is provided at the floor side end between the pair of flat plates, the bottom surface member defines the bottom surface in a convex shape directed vertically downward toward the center between the flat plates, and the gas introduction tube is provided above the lower end of the convex-shaped portion. With the above-described constitution, minute gas bubbles from the gas introduction tube move not only upward but toward the entire circumference of the cross-sectional circle of the gas introduction tube including the bottom surface side of the box structure. Therefore, the microorganism that settles when gas supply is temporarily stopped does not remain in the corner between the bottom portion and the sides, and is likely to move up when gas supply is resumed.

Preferred embodiments of the present invention also provide a bag for use in the culturing apparatus of the present invention, wherein a gas introduction tube having minute holes along the entire circumference of its substantially circular cross-section and a gas supply tube for supplying gas to the gas introduction tube are provided inside the bag, the bag is provided with only one opening for letting in the microorganism and the culturing solution, one end of the gas supply tube is connected to the gas introduction tube, and the other end of the gas supply tube extends to the outside through the opening.

The bag according to preferred embodiments of the present invention preferably has a substantially rectangular shape having longer and shorter sides, with the opening formed at one end of the shorter side of the rectangle, with the gas introduction tube disposed along the longer side of the rectangle on the other end side of the shorter side, and with shaft passage members for letting a shaft pass through provided along the longer side on the side the opening is provided.

Preferred embodiments of the present invention also provide a method of inserting a bag into a support of a culturing apparatus of the present invention (with the support being a box structure made up of the flat plates and the framework), the method including the steps of providing the bag according to a preferred embodiment of the present invention described above, passing a shaft longer than the longer side of the bag through the shaft passage members of the bag, turning the shaft to roll up the bag around the shaft with the gas introduction tube disposed in the lower portion of the bag substantially parallel to the shaft so that the portion of the bag that the gas introduction tube is disposed in is located on the outermost side and both ends of the shaft are exposed, then placing a roll body which includes the shaft and the bag above the framework, and rolling out the bag from the shaft to insert the bag into the framework.

This makes it possible to easily insert the bag of a preferred embodiment of the present invention into the support of the specific culturing apparatus of the present invention.

Preferred embodiments of the present invention also provide a microorganism culturing apparatus including a container for holding a microorganism and a culturing solution, and a gas introduction tube for introducing gas into the culturing solution in the container, wherein the gas introduction tube is provided with minute holes along an entire circumference thereof, with the bottom surface of the container having a convex shape facing vertically down toward the center of the width, with the gas introduction tube disposed above the lower end of the convex-shaped portion.

With the above-described arrangement, minute gas bubbles from the gas introduction tube move not only upward but toward the entire circumference of the cross-sectional circle of the gas introduction tube including the bottom surface side of the box structure. Therefore, a microorganism that settles when the gas supply is temporarily stopped does not remain in the corner between the bottom portion and the sides, and is likely to move up when gas supply is resumed.

Other features, elements, steps, characteristics and advantages will become more apparent from the following detailed description of preferred embodiments with reference to the attached drawings.

### BRIEF DESCRIPTION OF DRAWINGS

FIG. 1 is a front view showing a box structure of a culturing apparatus according to a first preferred embodiment of the present invention.

FIG. 2 is a plan view of the box structure of the culturing apparatus of the first preferred embodiment of the present invention.

FIG. 3 is a side view of the box structure for the culturing apparatus of the first preferred embodiment, as seen along the arrow A in FIG. 1.

FIG. 4 is a sectional view, taken along line B-B in FIG. 1.

FIG. 5 is a sectional view, taken along line C-C in FIG. 1.

FIG. 6 is a sectional view, taken along line D-D in FIG. 1.

FIG. 7 is a front view with flat plates removed from the box structure, shown in FIG. 1.

FIG. 8 is a front view of a bag for use in the first preferred embodiment of the present invention.

FIG. 9 shows the culturing apparatus of the first preferred embodiment with the bag filled with a solution put into the box structure, corresponding to a section E-E in FIG. 1.

FIG. 10 shows the culturing apparatus of the first preferred embodiment with the bag filled with a solution put into the box structure, corresponding to a section F-F in FIG. 1.

FIG. 11 is a front view corresponding to FIG. 7 with the flat plates attached to the framework and the posts.

FIG. 12 is a front view showing a box structure for a culturing apparatus according to a second preferred embodiment of the present invention.

FIG. 13 is a plan view showing the box structure for a culturing apparatus according to the second preferred embodiment of the present invention.

FIG. 14 is a sectional view taken along line G-G in FIG. 12.

FIG. 15 is a partial enlarged view showing the upper portion of FIG. 14.

FIG. 16 is a partial enlarged view showing the lower portion of FIG. 14.

FIG. 17 is a front view in the process of assembling the box structure of FIG. 12.

FIG. 18 is a front view showing a bag according to another preferred embodiment of the present invention.

FIG. 19 is a perspective view showing a method of inserting the bag.

FIG. 20 is a sectional view for explaining a method of spacing out both beam members, as an example of a method that is different from the method of the first and second preferred embodiments of the present invention.

### DETAILED DESCRIPTION OF PREFERRED EMBODIMENTS

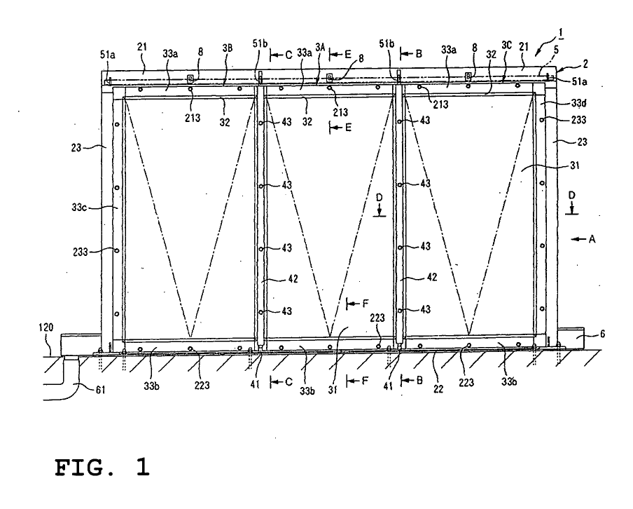

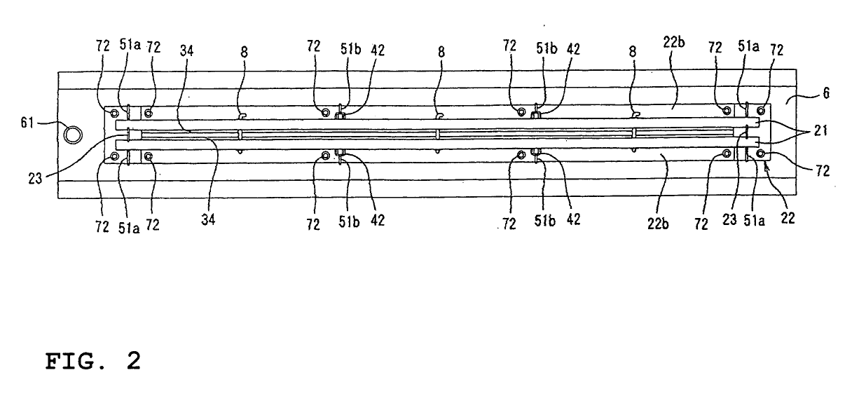

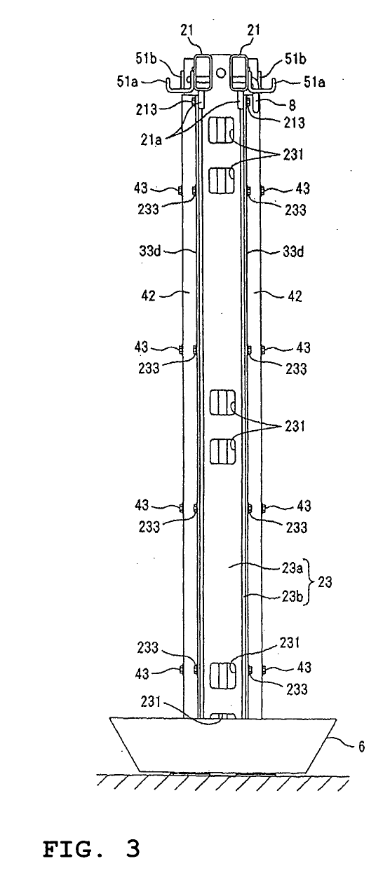

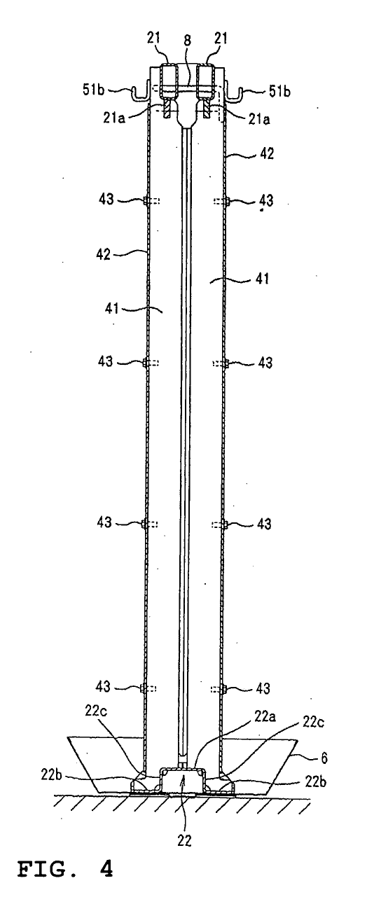

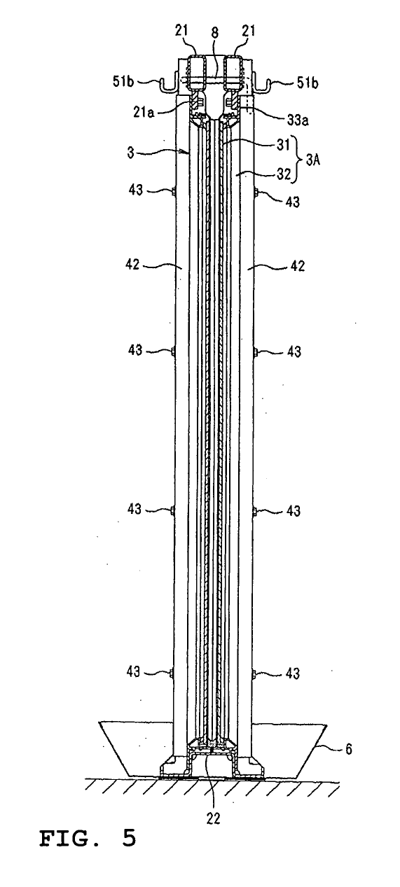

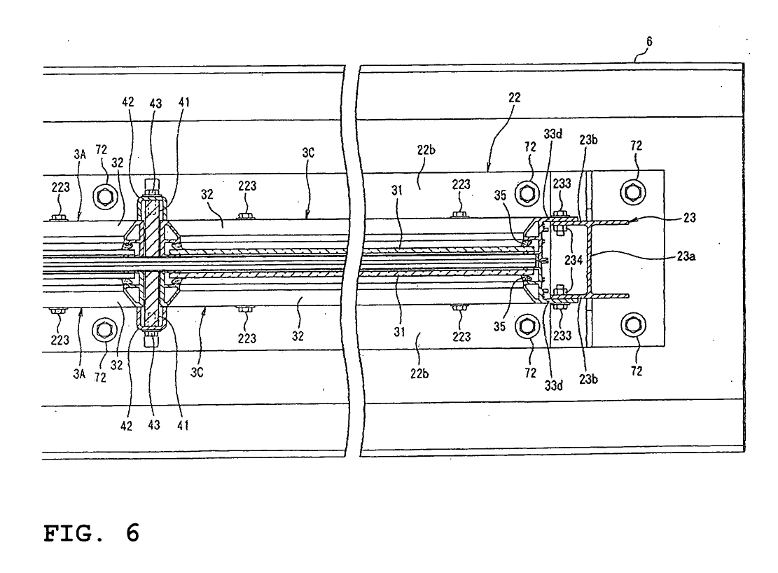

Preferred embodiments of the present invention are described below. A microorganism culturing apparatus according to a first preferred embodiment of the present invention is described with reference to FIGS. 1 to 11. The microorganism culturing apparatus of the present preferred embodiment preferably includes a bag as an example of a container for holding a culturing solution, and a box structure as an example of a support for supporting the bag. FIG. 1 is a front view of the box structure. FIG. 2 is a plan view of the box structure. FIG. 3 is a side view of the box structure, corresponding to the view as seen along the arrow A in FIG. 1. FIG. 4 shows a section B-B in FIG. 1. FIG. 5 shows a section C-C in FIG. 1. FIG. 6 shows a section D-D in FIG. 1.

As shown in FIG. 1, a box structure 1 preferably includes a framework 2 or a flattened parallelepiped skeleton, three pairs of substantially rectangular flat plates (first vertical surface members) 3A to 3C, and connection members (posts 41, pressing members 42, and bolts 43) interconnecting the central flat plate 3A and flat plates 3B and 3C on right and left sides of the central flat plate 3A. The box structure 1 is preferably installed in a predetermined outdoor position, with the lower portion of the framework 2 fixed to a horizontal surface 120 positioned on the ground.

The culturing apparatus of the present preferred embodiment also includes, in the upper portion of the framework 2, a pipe 5 for supplying temperature control water to front and back sides of the box structure 1. Hook members 51a and 51b for routing the pipe 5 are fixed to the upper portion of the framework 2. A gutter 6 for receiving water, supplied from the pipe 5 and falling down through the box structure 1, is provided in the lower portion of the box structure 1. A drain pipe 61 as shown in FIG. 2 is also provided to discharge water received within the gutter 6. In FIG. 1, the front side of the gutter 6 is removed to expose the lower portion of the box structure 1.

As shown in FIGS. 1 and 6, the flat plates 3A to 3C are preferably substantially rectangular, tempered glass plates 31 and aluminum frames 32 protecting the periphery of the glass plates 31. The surface of the tempered glass plate 31 is preferably coated with a thin film of photo-catalyst, titanium oxide (TiO2). This coating is provided to decompose soil on the surface of the tempered glass plate 31. Each of the flat plates 3A to 3C is continuously formed with an upper connection plate 33a at the upper end as an attachment portion for engaging with the framework 2 and with a lower connection plate 33b at the lower end as an attachment portion for engaging with the framework 2. The left end of the frame 32 of the left hand flat plate 3B is continuously formed with a side connection plate 33c as an attachment portion for engaging with the framework 2. The right end of the frame 32 of the right hand flat plate 3C is continuously formed with a side connection plate 33d as an attachment portion for engaging with the framework 2.

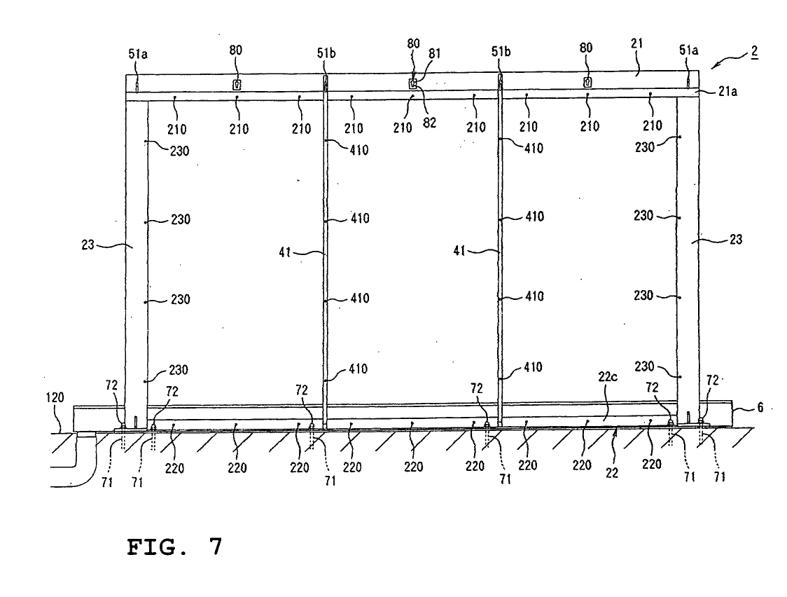

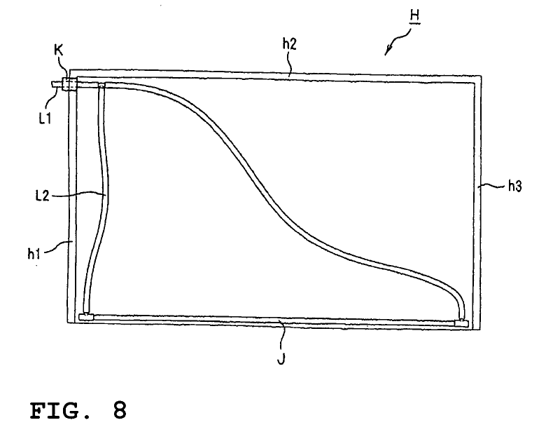

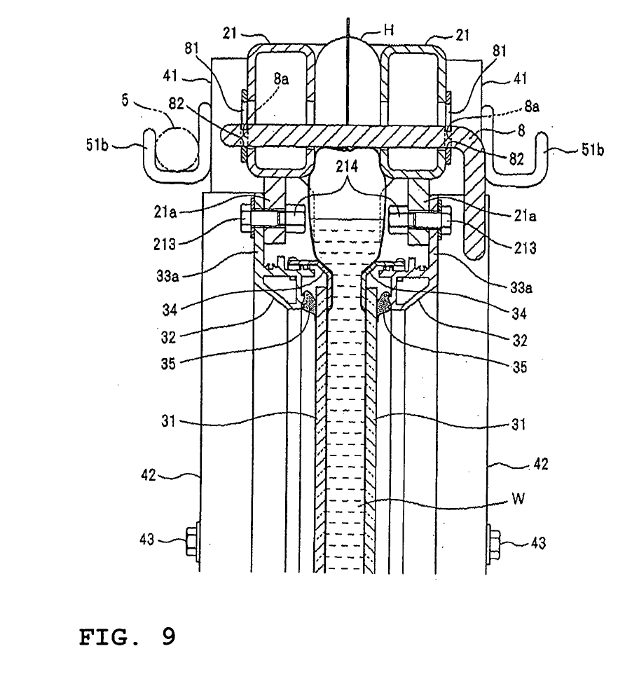

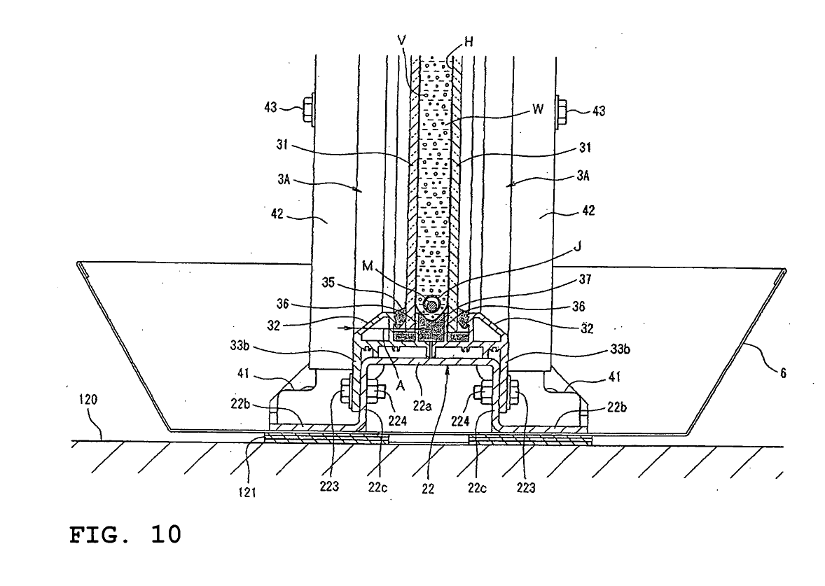

FIG. 7 is a front view with the flat plates 3A to 3C removed from the box structure 1 shown in FIG. 1. FIG. 8 is a front view of the bag for use in the first preferred embodiment of the present invention. FIGS. 9 and 10 show a state with the bag filled with solution put in the box structure, with FIG. 9 showing the section E-E and FIG. 10 the section F-F. As shown in these drawings, the framework 2 is preferably a substantially parallelepiped shaped member including a pair of beam members 21 forming its top surface, a floor member 22 as an example of the bottom member forming its lower surface, and a pair of side members (second vertical surface members) 23 forming the right and left side surfaces of the parallelepiped. As shown in FIG. 7, upper and lower ends of the posts 41 are fixed to the framework 2. As shown in FIG. 4, the top portions of the posts 41 are formed with blunt edges so as not to graze or puncture the bag when the bag is put in. Members corresponding to the “side plates” of this preferred embodiment are three pairs of flat plates or the first vertical surface members and the second vertical surface members. In other words, the support keeps the container filled with the culturing solution in a predetermined shape with the bottom portion (or bottom surface or lower portion), and the side plates defining the side surfaces.

As shown in FIGS. 7 and 9, the beam member 21 preferably includes a substantially square pipe, to the lower end of which is fixed a connection plate 21a for connection to the upper ends of the flat plates 3A to 3C. As shown in FIG. 9, upper connection plates 33a of the plates 3A to 3C are fixed to the connection plate 21a preferably using bolts 213 and nuts 214, for example. In other words, the connection plate 21a corresponds to the engagement portion (for engaging the paired flat plates with the framework through predetermined spacing) provided on the beam member.

Reference numeral “34” in FIG. 9 denotes a member for holding from inside the upper periphery of the tempered glass plates 31 of the flat plates 3A to 3C and having blunt edges so as not to graze or puncture the bag H. Reference numeral “35” indicates a seal member arranged to fill up the gap between the frame 32 and the tempered glass plates 31. The floor member 22 is preferably made of steel and has a rail shaped configuration, with its cross section as shown in FIG. 10 integrally including a central horizontal plate 22a in the center of the width, horizontal end plates 22b located lower than the central horizontal plate 22a and extending sideways, and vertical plates 22c interconnecting the former two. The horizontal end plates 22b are provided with through holes to be fixed to the ground. The central horizontal plate 22a is disposed the lower end of the frame 32. The lower connection plates 33b of the flat plates 3A to 3C are fixed to the outer side of the vertical plates 22c preferably using bolts 223 and nuts 224. In other words, the vertical plates 22c correspond to the engagement portions (for engaging the paired flat plates with the framework through predetermined spacing) provided on the floor member.

Reference numeral “36” in FIG. 10 denotes a member for holding from inside the lower periphery of the tempered glass plate 31 of the flat plates 3A to 3C, formed integrally with the frame 32 holding the tempered glass plates 31 from outside through the seal member 35. Reference numeral “37” denotes the bottom surface member fixed to one of the holding members 36 of the flat plates 3A to 3C disposed opposite to each other. The side member 23 includes an H-section steel member, as shown in FIGS. 3 and 6, including a web 23a and flanges 23b. As shown in FIG. 6, side connection plates 33c and 33d of the flat plates 3B and 3C are fixed with bolts 233 and nuts 234 to portions of the flanges 23b located more inwardly than the web 32a inside the framework 2. As shown in FIG. 3, the side member 23 is provided with substantially rectangular openings 231 at positions spaced at intervals in the longitudinal direction of the web 32a.

The framework 2 as shown in FIGS. 6 and 7 is provided with through holes 210, 220 and 230 for connecting the flat plates 3A to 3C, respectively in the connection plate 21a of the beam member 21, in the vertical web 22c of the floor member 22, and in the flange portion 23b of the side member 23. The post 41 is provided with female threads 410. Further, as shown in FIGS. 3, 4 and 5, hook members 51a are fixed to the beam member 21 located above the side member 23. Hook members 51b are fixed to the upper portion of the post 41.

Keyholes 80 for inserting pins 8 are provided in opposing positions of the beam members 21. The keyhole 80 as shown in FIG. 7 includes a substantially round hole 81 having a diameter that is slightly greater than the diameter of the cross-sectional circle of the pin 8 and a substantially square opening 82 continuing downward from the round hole 81. As shown in FIG. 9, the pin 8 is provided with grooves 8a, spaced apart from each other by a specified distance, to engage with the substantially square opening 82. The bag H is preferably transparent and substantially rectangular as shown in FIG. 8 with an opening K provided at one end of the shorter side of the rectangle. In the bag H are disposed a gas introduction tube J and gas supply tubes L1 and L2 connected to both ends of the gas introduction tube J. The gas introduction tube J is preferably provided with minute holes over its entire cross-sectional circle and may be a commercially available one such as that manufactured by UNIHOSE Co., Ltd., of porous rubber under the tradename of “SEEPERHOSE.” As shown in FIG. 10, a rod-shaped weight M is preferably inserted in the gas introduction tube J. The gas introduction tube J is disposed in the bag H along its one longer side opposite to the side where the opening K is provided.

The gas supply tubes L1 and L2 are provided to supply gas to both ends of the gas introduction tube J and may be commercially available hoses made of soft plastic. The gas supply tube L2 is connected to the gas supply tube L1 near the opening K in the bag H, with the gas supply tube L1 only extending out of the bag H. The bag H may be made, for example, by folding a transparent, soft, plastic sheet (made of polyethylene, polypropylene, and polyvinyl chloride, for example) in two and welding along three open sides h1 to h3 of the folded sheet. The gas introduction tube J and the gas supply tubes L1 and L2 in the interconnected state are put in the bag H so that the gas supply tube J projects out through the opening K.

To assemble the box structure 1, first as shown in FIGS. 7 and 10, the floor member 22 of the framework 2 is fixed to a horizontal surface (ground) 120 through a butyl rubber-made sheet 121 and a gutter 6. To fix the framework 2, anchor bolts 71 and cap nuts 72 are preferably used, although other suitable fixing elements may be used. The horizontal end plates 22b of the floor member 22 are provided with through holes for the anchor bolts 71 to pass. Next, the side members 23 are disposed at both longitudinal ends of the floor member 22, and the lower portion of the side members 23 are fixed to the floor member 22 preferably using bolts (not shown) or other fixing elements. Then, a pair of beam members 21, having a predetermined spacing in between, are disposed over both of the side members 23. In this state, the upper portions of the side members 23 and the beam members 21 are fixed together preferably using bolts (not shown) or other fixing elements. As a result, the framework 2 is installed on the horizontal surface (ground) 120 in the state shown in FIG. 7.

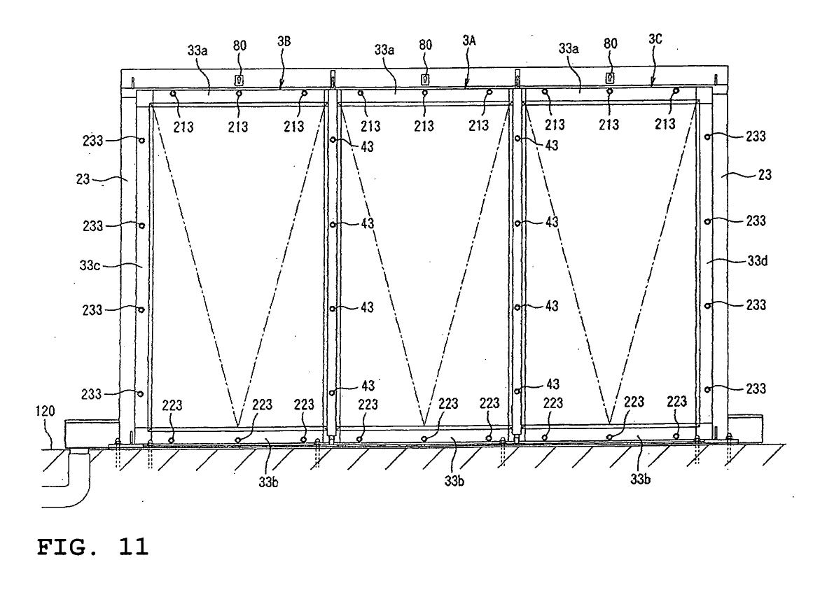

Next, the flat plates 3A to 3C are attached to the framework 2 and the posts 41 in the state shown in FIG. 7 to bring about the state shown in FIG. 11. In other words, as shown in FIGS. 9 and 11, the upper connection plates 33a of the flat plates 3A to 3C are mated with the upper connection plates 21a of the framework 2 and fastened together preferably using bolts 213 and nuts 214. As shown in FIGS. 10 and 11, the lower connection plates 33b of the flat plates 3A to 3C are mated with the lower connection plates 22c of the framework 2 and fastened together preferably using bolts 223 and nuts 224. As shown in FIGS. 1 and 11, the left side connection plates 33c of the flat plate 3B is mated with the side member 23 on the left side of the framework 2 to fasten them together preferably using bolts 233 and nuts 234. As shown in FIGS. 1, 6, and 11, the right side connection plate 33d of the flat plate 3C is mated with the side member 23 on the right side of the framework 2 to fasten them together preferably using bolts 233 and nuts 234.

In the above-described state, as shown in FIG. 6, the pressing member 42 is engaged with the left edge of the flat plate 3C located on the right side of the post 41 and with the right edge of the flat plate 3B located on the left side of the post 41, and bolts 43 are screwed from outside in the normal direction to the side plates into the female threads provided in the post 41. The flat plate 3B and the flat plate 3A are likewise fixed to the post 41. In the box structure 1 assembled as described above, a space is formed by the mutually opposing flat plates 3A to 3C and the side members 23. The bottom of the space is formed as shown in FIG. 10 with the bottom surface member 37 having a convex shape directed vertically down toward the center between the flat plates.

Next, after inserting the bag H from above into the box structure 1, the pins 8 are fit in the key holes 80. In other words, each pin 8 is inserted into the substantially round hole 81 of the key hole 80 to pass through both of the beam members 21, and the groove 8a of each pin 8 is made to fit in the substantially square hole 82 of each beam member 21. By the engagement of the grooves 8a of the pins 8 with the substantially square holes 82, the distance between the beam members 21 is kept constant at three positions in the lengthwise intermediate portion (excluding both ends).

Next, the gas supply tube L1 extending out through the opening K of the bag H is connected to a gas supplier. Then, after putting a predetermined amount of sterile culturing solution through the opening K into the bag H, a microorganism to be cultured is put into the bag H, and the bag H is filled with liquid W up to a predetermined level in the bag H as shown in FIG. 9. In this state, as shown in FIG. 10, the gas introduction tube J with the weight M is disposed near the bottom surface member 37.

Next, culture is started by supplying temperature control water from the pipe 5 to front and back sides of the box structure 1 and supplying gas to the gas supply tubes L1 and L2. Along with this, minute gas bubbles V are supplied from the gas introduction tube J into the entire liquid W and sunlight coming through the flat plates 3A to 3C or the tempered glass plates 31 enters the liquid W to carry out the culture of microorganism. At the time of this culture, the pressure of the liquid W in the bag H exerts forces in the direction of widening the distance between the beam members 21 and between the paired flat plates 3A to 3C. However, because the engagement between the grooves 8a of the pins 8 and the substantially square holes 82 at the longitudinally spaced three points along the intermediate portion of the beam members 21 maintains the distance between the beam members 21, the beam members 21 and the flat plates 3A to 3C are prevented from warping outward. Although the gas supplied into the liquid W collects in the upper portion of the bag H, it is discharged through the opening K. In case of emergency, the solution in the bag H may be discharged by piercing a hole in the bag H with a pointed tool inserted through the opening 231 of the side member.

When the culture is complete, a pump is connected to the opening K to retrieve the liquid W from the bag H, pins 8 are removed, and the bag H is taken out through the upper portion of the box structure 1. If another bag H is used for the next culture, cleaning of the container is not necessary and may be omitted. Moreover, if cleaning of the flat plates 3A to 3C is to be done, it can be done easily by taking them out of the framework 2 after removing the bolts 213, 223, 233 and 43.

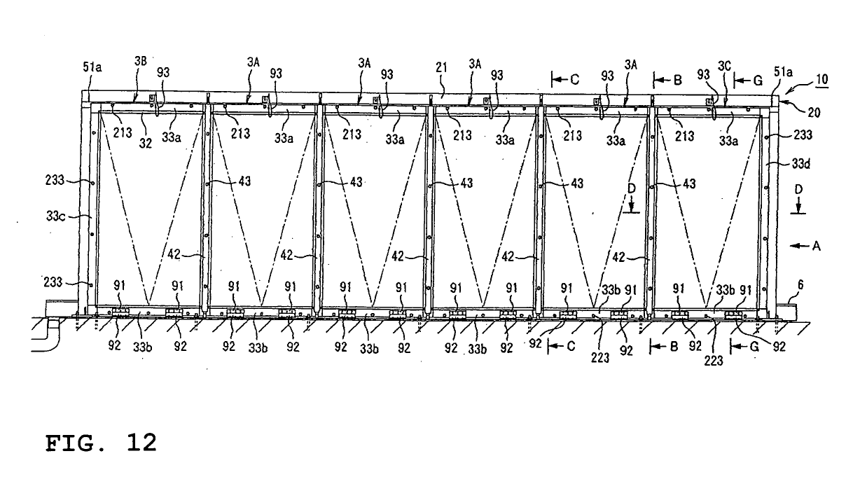

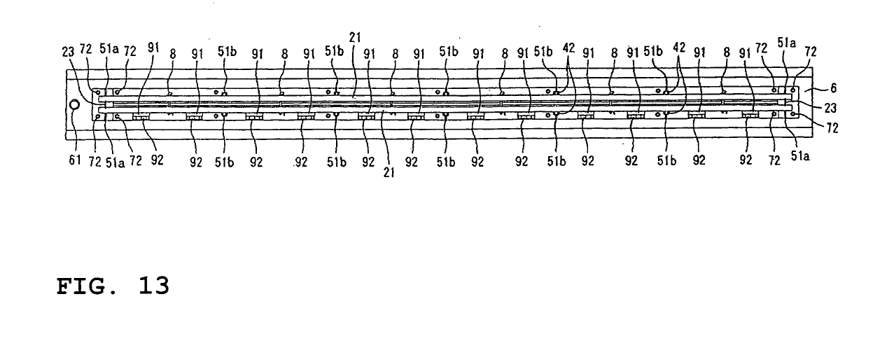

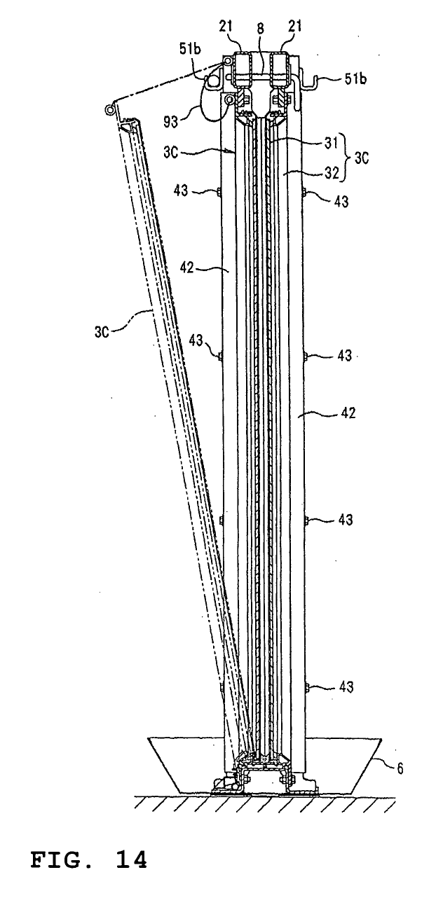

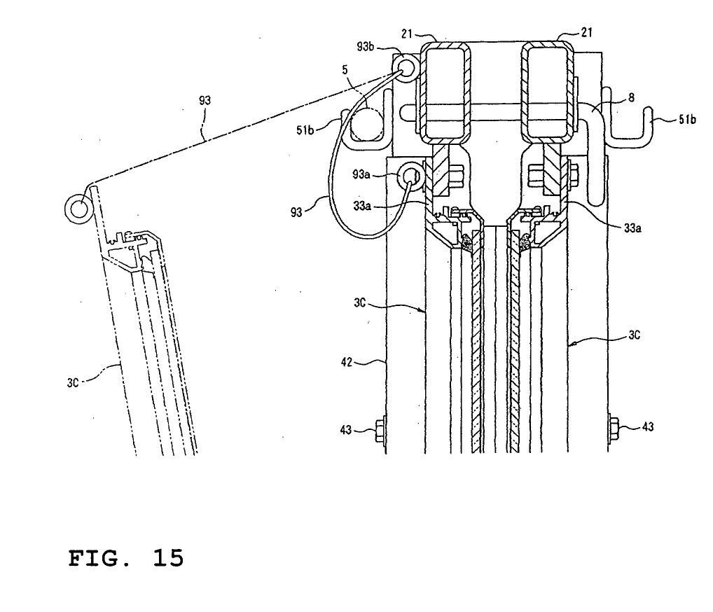

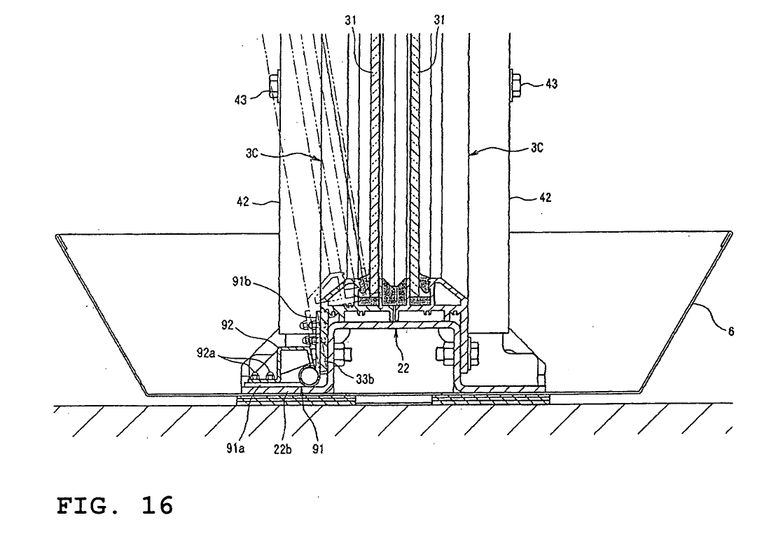

Referring to FIGS. 12 to 17, a microorganism culturing apparatus according to a second preferred embodiment of the present invention is described. The culturing apparatus of the present preferred embodiment is provided with a box structure 10 as the support for supporting the bag. FIG. 12 is a front view of the box structure 10. FIG. 13 is a plan view of the box structure 10. FIG. 14 is a sectional view, taken along line G-G in FIG. 12. FIG. 15 is a partial enlarged view of the upper portion of FIG. 14. FIG. 16 is a partial enlarged view of the lower portion of FIG. 14. Incidentally, the view of the section B-B in FIG. 12 is the same as that in FIG. 4, the section C-C in FIG. 12 is the same as that in FIG. 5, and the section D-D in FIG. 12 is the same as that in FIG. 6.

The culturing apparatus of the second preferred embodiment is similar to that of the first preferred embodiment, with differences in the following points described below. As shown in FIG. 12, the box structure 10 preferably includes six pairs of the flat plates 3A to 3C. Therefore, a framework 20 is used that is twice as wide as the framework 2 of the first preferred embodiment. Further, as shown in FIGS. 12 and 16, the lower connection plates 33b of the flat plates 3A to 3C are connected through hinges 91 to the horizontal end plates 22b of the floor member 22. In other words, one plate 91a of the hinge 91 is fixed to the horizontal end plate 22b and the other plate 91b is fixed to the lower connection plate 33b. To restrict the rotation range of the flat plates 3A to 3C permitted with the hinges 91, stoppers 92 are fixed with bolts 92a to the one plate 91a.

Further as shown in FIGS. 12 and 15, the upper ends of the flat plates 3A to 3C and the beam members 21 of the framework 2 are interconnected through wires (linear members) 93 of a predetermined length. As shown in FIG. 15, a ring 93a for fixing one end of the wire 93 is fixed to the upper connection plate 33a of each of the flat plates 3A to 3C. A ring 93b for fixing the other end of the wire 93 is fixed to each of corresponding positions on the beam member 21.

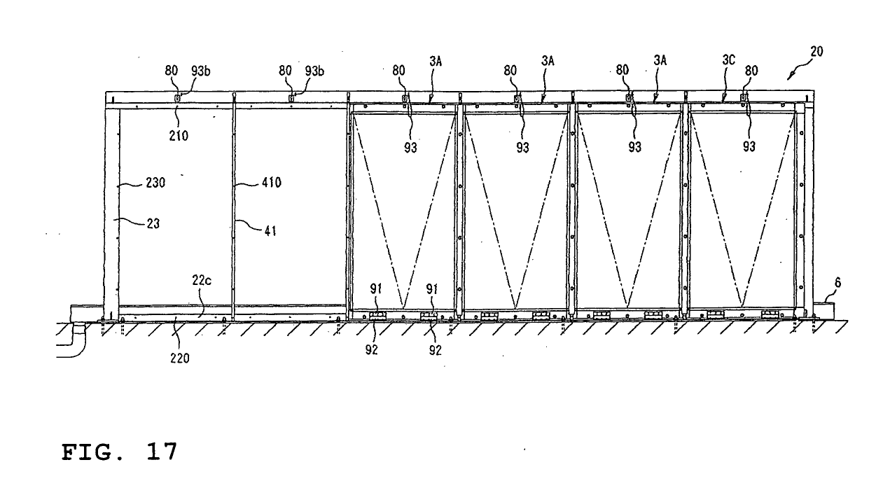

FIG. 17 is a front view in the process of assembling the box structure 10 in the state of four flat plates 3A, 3C on the right hand attached to the framework 20 fixed to the ground. In other words, in the second preferred embodiment, the flat plates 3A to 3C are attached to the framework 2 in the same manner as in the first preferred embodiment, and then the hinges 91, the stoppers 92, and the wires 93 are attached. With the microorganism culturing apparatus of the second preferred embodiment, the following effects and advantages are obtained in addition to those obtained with the microorganism culturing apparatus of the first preferred embodiment.

With the bolts 213, 223, 233 and 43 removed, the lower portions of the flat plates 3A to 3C may rotate with the hinges 91 and their upper portions may open. At this time, the rotation range of the flat plates 3A to 3C is restricted by the stoppers 92, so that the upper portions of the flat plates 3A to 3C open according to the length of the wires 93. Because cleaning of the flat plates 3A to 3C may be done in the above-described state, cleaning of the flat plates 3A to 3C may be performed more easily in comparison with the culturing apparatus of the first preferred embodiment.

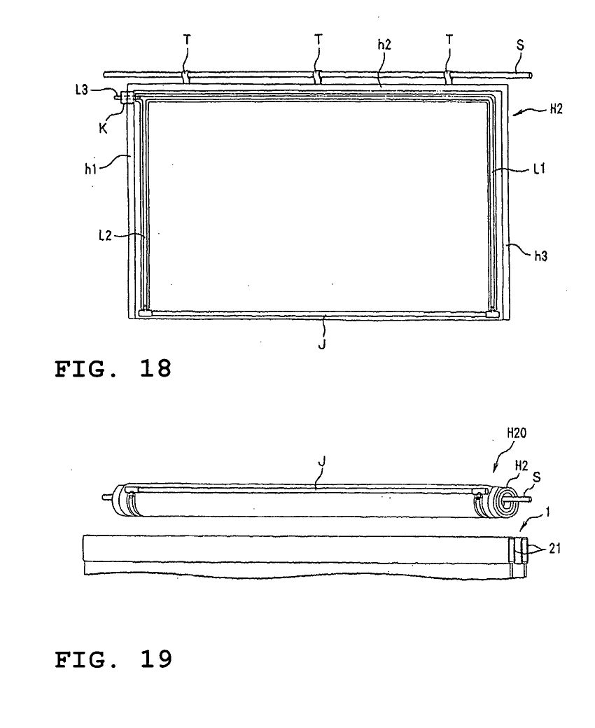

Furthermore, because six pairs of the flat plates 3A to 3C are preferably provided, the volume for culture is twice that of the culturing apparatus of the first preferred embodiment. Referring to FIGS. 18 and 19, a method of inserting the bag according to this preferred embodiment of the present invention is described. In the method of this preferred embodiment, a bag H2 having the shape shown in FIG. 18 is preferably used. The bag H2, like that shown in FIG. 8, is preferably transparent and substantially rectangular, with the opening K provided at one end of one shorter side of the rectangle. In the bag H2 are disposed the gas introduction tube J and gas supply tubes L1 and L2 connected to both ends of the gas introduction tube J. The gas introduction tube J is preferably substantially the same as that used in the bag H shown in FIG. 8. The gas supply tubes L1 and L2 are more flattened in cross-section than that used in the bag H shown in FIG. 8. Further, the opening K is provided with a gas introduction inlet L3 extending out from the opening K of the bag H.

The bag H2 is also provided with rings T (shaft passage members), which are made by cutting out from a plastic sheet, along the longer side on the side the opening K is disposed. As shown in FIG. 19, a shaft S longer than the longer side of the substantially rectangular bag H2 is passed through the rings T, with a roll H20 formed by rolling the bag H2 around the shaft S disposed over the box structure 10. To roll the bag H2, the shaft S is turned with the gas introduction tube J put inside lower portion of the bag H2 parallel to the shaft S, so that the portion containing the gas introduction tube J is on the outermost side and that both ends of the shaft S are exposed.

In the state of FIG. 19, the shaft S is supported and the roll of bag H2 is rolled out and put into the box structure 1 through between the beam members 21. While the flat plates 3A to 3C, each including the tempered glass plate 31 attached to the aluminum frame 32, are preferably used in the above preferred embodiments, the tempered glass plate is not a limitation but mesh or lattice may be used as long as it suffices to support the bag H in the predetermined shape. Further, while the bottom member 22 disposed on the horizontal surface 120 is preferably used in the above-described preferred embodiments, it is also possible to use the horizontal surface 120 directly as the bottom of the support.

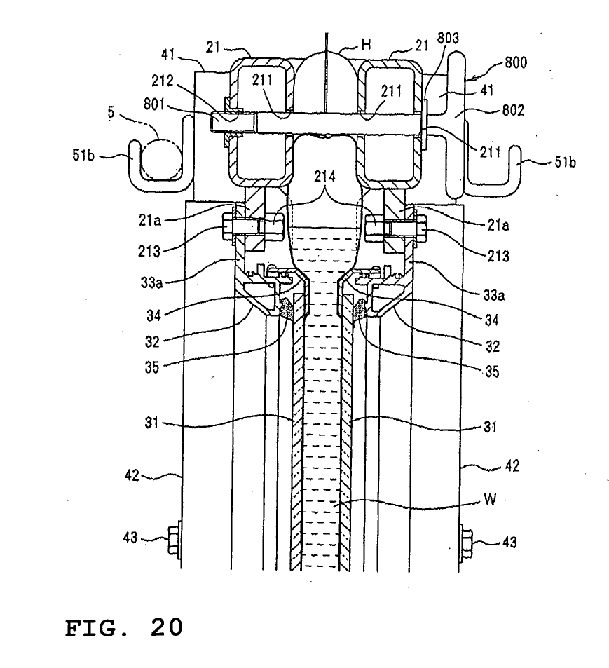

In the above-described preferred embodiments, the spacing between the beam members 21 is kept unchanged by fitting the pin 8 to the keyhole 80 of the framework 2. An example of another method of maintaining constant spacing is described below. In this example, as shown in FIG. 20, the pin 800 is provided with a male thread 801 at one longitudinal end thereof, and with a handle 802 and a washer 803 at the other end thereof. The beam members 21 of the framework 2 are provided with through holes 211 for passing the pin 800, with one beam member 21 provided with a female thread 212 for screw engagement with the male thread 801 of the pin 800. Holding the handle 802 by hand, the pin 800 is passed into the through hole 211 of the beam member 21, the handle 802 is turned, the fore-end male thread 801 is engaged with the female thread 212 of the beam member 21, and the washer 803 is made to contact the beam member 21, to maintain spacing between the beam members 21.

Further, if the flat plates 3A to 3C of different dimensions A (See FIG. 10) in the thickness direction of the frame 32 are prepared in advance to cope with a plural number of light paths, the light path may be easily changed by simply replacing the flat plates 3A to 3C. In this way, for example, a single culturing apparatus may be used both in a culture intended for mass production with the light path set to a maximum according to the culturing conditions and in a culture with a small light path for subject microorganism that requires intense incident light.

The microorganisms to be cultured with the microorganism culturing apparatus of various preferred embodiments of the present invention are tiny living things that cannot be observed by the naked eye. Specific examples of such microorganisms may include prokaryotes, and eukaryotes such as protozoa, yeast, filamentous fungi, myxomycetes, basidiomycetes and microalgae. The microorganism culturing apparatuses according to various preferred embodiments of the present invention are appropriate for culturing photosynthetic microorganisms that carry out photosynthesis by taking sunlight or artificial light into the culturing space, and are particularly appropriate for microalgae.

While many photosynthetic microorganisms utilize infrared rays for the photosynthesis, the microalgae mainly utilize visible radiation for the photosynthesis and not infrared radiation. Because the infrared radiation is heat radiation, casting light to the culturing solution, with the infrared radiation removed, makes it possible to restrict temperature increases in the culturing solution. Therefore, microalgae may be efficiently cultured with the culturing apparatus of preferred embodiments of the present invention by casting mainly visible radiation into the container to prevent temperature increases in the culturing solution while blocking the entry of infrared radiation.

Methods of casting the visible radiation into the culturing apparatus of preferred embodiments of the present invention while blocking the entry of the infrared radiation may include making the side plates or the container with a material that permits entry of visible radiation while preventing entry of infrared radiation, placing a filter that permits entry of visible radiation while preventing entry of infrared radiation on the inside or outside surface of the side plates or the container, or using a light source that emits visible radiation but does not emit infrared radiation.

As described above, the microorganism culturing apparatus of preferred embodiments of the present invention, including the container for holding a culturing solution and the support for accommodating the container, is capable of restricting multiplication of unwanted miscellaneous germs and producing the same identical culturing space repeatedly. Further, by making the bag (the container) as a throwaway bag, miscellaneous germs are made less likely to grow even if cleaning is not done every time.

While the present invention has been described with respect to preferred embodiments, it will be apparent to those skilled in the art that the disclosed invention may be modified in numerous ways and may assume many embodiments other than those specifically set out and described above. Accordingly, it is intended by the appended claims to cover all modifications of the present invention which fall within the true spirit and scope of the present invention.

1-16. (canceled)

17. A microorganism culturing apparatus comprising:
a microorganism;
a culturing solution for culturing the microorganism;
a container arranged to hold the culturing solution; and
a support arranged to support and hold the container; wherein
the container takes on and maintains a predetermined shape by being supported by the support.

18. The culturing apparatus of claim 17, wherein the support includes a bottom member defining a bottom surface and side plates defining side surfaces, the side plates and the container having transparent portions, respectively, and the transparent portions of the side plates and the transparent portions of the container overlap each other.

19. The culturing apparatus of claim 18, wherein the side plates are made of flat plates and at least one set of paired flat plates oppose each other.

20. The culturing apparatus of claim 17, wherein the support includes side plates defining side surfaces and a framework arranged to support the side plates, and end portions of the side plates have attachment portions that are arranged to engage with the framework and that are removably fixed to the framework.

21. The culturing apparatus of claim 20, further comprising posts disposed upright with both ends thereof fixed to the framework, and pressing members that are disposed outside of and substantially parallel to the posts, wherein the side plates are squeezed between the pressing members and the posts, and the pressing members are fixed to the posts.

22. The culturing apparatus of claim 21, wherein the side plates are flat plates disposed end to end in a longitudinal direction of the framework, the flat plates are interconnected through the posts end to end in the longitudinal direction of the framework, with two adjacent ends of the flat plates fixed to one post with one pressing member.

23. The culturing apparatus of claim 17, further comprising a gas introduction tube arranged to introduce gas into the culturing solution, the gas introduction tube is provided in the container.

24. The culturing apparatus of claim 20, wherein the side plates are supported for rotation about bottom ends in a state of fixation between the attachment portions and the framework being released.

25. The culturing apparatus of claim 24, further comprising wires, wherein the ends of the side plates on the upper portion side of the framework are connected through the wires to the upper portions of the framework.

26. The culturing apparatus of claim 24, further comprising a stopper that is provided at a bottom portion of the framework to restrict the rotation range of the side plates.

27. The microorganism culturing apparatus of claim 20, wherein the framework and the sides plates are movable relative to each other in a state of the fixation between the attachment portions and the framework being released, and the relative motion produces a clearance between the upper portion of the framework and the ends of the side plates on the upper portion side of the framework.

28. A microorganism culturing apparatus for culturing microorganism in a culturing solution, comprising:
side plates defining side surfaces of the culturing apparatus and arranged to define a culturing space; and
a bottom portion defining a bottom of the culturing apparatus to accommodate the culturing solution; wherein
the bottom portion has a convex shape directed vertically down toward a center of the width of the culturing apparatus, and a gas introduction tube arranged to introduce a gas into the culturing solution is disposed above the bottom portion at a lower end of the convex-shape of the bottom portion.

29. The culturing apparatus of claim 28, wherein the gas introduction tube is provided with holes along an entire cross-section of a substantially circular circumference thereof.

30. The culturing apparatus of claim 17, wherein the container includes therein a gas introduction tube having minute holes along an entire cross-section of a substantially circular circumference thereof and a gas supply tube arranged to supply gas to the gas introduction tube, and the container further includes an opening at only one position for putting in the microorganism and the culturing solution, with one end of the gas supply tube connected to the gas introduction tube and with the other end of the gas supply tube extending out through the opening.

31. The culturing apparatus of claim 30, wherein the container is a substantially rectangular bag having longer sides and shorter sides, the opening is provided at one end portion of the shorter side of the substantially rectangular bag, the gas introduction tube is disposed at the other end portion of the shorter side of the substantially rectangular bag along the longer side of the substantially rectangular bag, and shaft passage members arranged to allow a shaft to pass through are provided along the longer side on the side on which the opening is provided.

32. A method of culturing a microorganism comprising the steps of:
providing a microorganism;
providing a culturing solution for culturing the microorganism;
providing a container arranged to hold the culturing solution; and
providing a support arranged to support and hold the container; and
providing the container on the support such that the container takes on and maintains a predetermined shape by being supported by the support.

33. The method according to claim 32, further comprising the step of putting the microorganism and the culturing solution in the container before or after the step of providing the container on the support.

34. The method according to claim 32, wherein the container is a substantially rectangular bag having longer sides and shorter sides, an opening is provided at one end portion of the shorter side of the substantially rectangular bag, a gas introduction tube is disposed at the other end portion of the shorter side of the substantially rectangular bag along the longer side of the substantially rectangular bag, and shaft passage members arranged to allow a shaft to pass through are provided along the longer side on the side on which the opening is provided.

35. The method according to claim 34, further comprising the step of passing a shaft longer than the longer side through the shaft passage members of the bag, and turning the shaft to roll up the bag around the shaft with the gas introduction tube disposed in the lower portion of the bag substantially parallel to the shaft so that the portion of the bag that the gas introduction tube is disposed in is located on an outermost side and both ends of the shaft are exposed outside of the bag.

36. The method according to claim 35, wherein the support includes side plates defining side surfaces and a framework arranged to support the side plates, and end portions of the side plates have attachment portions that are arranged to engage with the framework and that are removably fixed to the framework, the method further comprising the steps of placing a roll body including the shaft and the bag above the framework, and rolling out the bag from the shaft to insert the bag into the framework.
